(19) United States
(12) Patent Application Publication
(10) Pub. No.: US 2025/0093328 A1
(43) Pub. Date: Mar. 20, 2025

(54) MEDICAL ANALYZER AND DIAGNOSTIC SAMPLE PROFILER

(71) Applicant: iFirst Medical Technologies, Inc., Honolulu, HI (US)

(72) Inventor: Luke B. Joseph, Honolulu, HI (US)

(21) Appl. No.: 18/812,185

(22) Filed: Aug. 22, 2024

Related U.S. Application Data
(63) Continuation of application No. 17/262,111, filed on Jan. 21, 2021, now abandoned.
(60) Provisional application No. 62/703,390, filed on Jul. 25, 2018.

(51) Int. Cl. G01N 33/49; G01N 11/14

(52) U.S. Cl. CPC G01N 33/4915; G01N 11/14; G01N 33/4905; G01N 2011/147

(57) ABSTRACT

A medical analyzer and coagulation profiler performs various interrogations on specimens. A motor with reduction gearing moves and a video camera observes the samples, the cartridges or parts thereof. Changes in images are compared and recorded with a central processor that controls a display. Power supply, temperature controller, motor and gearing are mounted in a box which attaches to a smartphone. The smartphone provides the video camera, illumination and central processor that control the movement, temperature and display. The device makes testing simpler for small hospitals, clinics, ambulances, remote locations and individuals and controls a number of parallel or serial devices operating simultaneously or sequentially. A cartridge insertion actuates a circular motion to generate a blood profile based on changes. Change is analyzed with a video camera and processor such as in a smartphone and is plotted to show an amplitude and time. A smartphone provides a specific movement pattern.

This application is a continuation of U.S. patent application Ser. No. 17/262,111, filed Jan. 21, 2021, which is the U.S. National Phase under 35 U.S.C. § 371 of International Application No. PCT/US2019/043400, filed Jul. 25, 2019, titled MEDICAL ANALYZER AND DIAGNOSTIC SAMPLE PROFILER, which claims the benefit under 35 U.S.C. § 119 as a nonprovisional of U.S. Prov. App. No. 62/703,390 filed on Jul. 25, 2018, which is hereby incorporated by reference in its entirety.

### BACKGROUND

Five million people around the world die of trauma on an annual basis. Up to 20% of these deaths are preventable with better control bleeding. In these types of traumatic injury, the incidences of coagulation abnormalities are high. For example, natural supplies of proteins such as Factor VII are quickly depleted after trauma, which can quickly lead to hemorrhage-related death. Detecting these abnormalities quickly after the trauma often can be a predictor of the patient's mortality. These diagnostics can be a decision aid for providers and provide feedback for lifesaving actions, such as transfusions.

Although techniques such as prothrombin time (PT) and partial thromboplastin time (PTT) can test coagulation, only the first state of coagulation and plasma hemostasis are tested rather than coagulocompetence. In addition it has been shown that PT and PTT tests do not predict coagulation abnormalities as effectively as coagulation profiles, such as thromboelastography (TEG) shown in FIG. 10A. In addition separating the plasma complicates the blood processing and adds steps to the coagulation initiation.

Other coagulation profiling techniques such as thromboelastography and rotational thromboelastometry (ROTEM) shown in FIG. 10B provide a more complete coagulation profile by using whole blood. The use of whole blood includes the role of platelets, blood factors and phospholipids in the coagulation cascade. Unfortunately both standard coagulation tests (PT, PTT, etc.), and newer systems such as TEG and ROTEM, require relatively large equipment, controlled conditions and trained technicians to perform tests. These limitations prevent these diagnostic tools from being at the point of injury (POI).

In order to most effectively treat traumatic injuries, it can be important to diagnose coagulation abnormalities at the POI, ideally by first responders such as paramedic and emergency medical technicians (EMT) (FIG. 9) Paramedics and EMTs could rapidly evaluate the coagulopathy and obtain guidance in using blood products or administration of coagulation related drugs. In addition, further integration of other coagulation relevant assays, such as complete blood count (CBC) or hematocrit (HTC), base deficit, platelet count, and Pa02 with a TEG-like profile could be an invaluable addition to point-of-care diagnostics.

Needs exist for improved base medical analyzers and coagulation profilers.

### SUMMARY

Some embodiments of the invention can solve the existing problems by providing new base medical analyzers and coagulation profilers that can be available to be quickly used.

An example of embodiments of the invention can include a new cartridge based biological microelectromechanical system (BioMEMS) that rotates back and forth in a circular motion in direct contact to a blood sample, while the blood coagulates. This rotation changes over time as the blood coagulates in the sample. The change in motion is analyzed through a video camera (in one case an iPhone camera) and then is plotted to show an amplitude over time. The plot of motion over time is indicative of particular forms of coagulation disorders. The rotating motion of the BioMEMS device is induced externally using a magnetic field. The rotation induced is not limited to a magnetic field but could be direct mechanical or electrostatic inducer of the rotation. The magnetic actuation is provided by a motor, servo or similar device that turns a magnet. The motor can be controlled mechanically or electronically, by the iPhone for example, to provide a specific pattern. In one case the pattern is about 4°45′ in 5 seconds. There can be a large range of patterns, dependent on application. In one case the profile is measured for about 30 to 60 minutes, however, time may vary depending on application.

Use of a mobile device, such as an iPhone and the new device has been demonstrated to show coagulation over time in the form of a coagulation profile. Some embodiments of the invention make the testing simpler by use of a cartridge and provides a method of having a large number of sequential tests to monitor a patient from POI to the emergency room (ER), operating room and recovery. The overall system and the cartridge can be very small. The use of cartridges in some embodiments of the invention simplifies the process as compared to conventional techniques. Being small and portable there is potential provided by some embodiments of the invention for a large number of parallel or serial devices operating simultaneously.

The system can comprise in some embodiments a handheld medical analyzer platform, which works with different disposable application cartridges to perform a variety of interrogations on specimen samples. One application includes attaching a biological microelectromechanical system (BioMEMS) cartridge that generates blood coagulation profiles indicative of particular forms of coagulation disorders. The device makes coagulopathy testing simpler for small hospitals, clinics, ambulances, remote locations and individuals by use of a cartridge and permits for a larger number of parallel or serial devices operating simultaneously. One insertion of a cartridge actuates an oscillating circular motion to generate a blood coagulation profile based on a change in rotational motion as blood coagulates in a sample. Change in rotational motion is analyzed through a video camera such as in a smartphone and is plotted to show an amplitude over time. Actuation of the BioMEMS can be achieved by magnetic actuation of a motor controlled by an iPhone or a smart phone to provide a specific rotational pattern.

A liquid coagulation measuring device can include a case and a motor within the case. Gearing can be connected to the motor. A magnet can be connected to the gearing and is configured for magnetic coupling to a movable element within a liquid well. A temperature controller can be connected to the case and can be configured for controlling temperature of liquid in the liquid well.

A light source illuminates the movable element and a recorder records movement of the movable element. A compact microscope can be configured for alignment with the liquid well and a video camera can be aligned with the compact microscope.

An attachment on the case can be configured for attaching to a smartphone having a video camera, a central processor and display. The attachment can be configured for aligning the video camera with the liquid well and the movable object.

The case can have a base and a cover. The base can have a bottom, sides and a top and a space in the top for positioning and holding a smartphone. The cover can be configured for covering border areas around a display face of the smartphone. The cover and the sides have complementary connections configured for holding the cover on the base and holding the smartphone within the case. One of the sides has an opening for receiving a cartridge with the well.

An elastomeric boot can surround the case and can be adapted for protecting the measuring device and the smartphone. The opening in the case can be configured for receiving the cartridge. A passage flows the liquid into the well through a cartridge port outside of the opening into one of the sides of the case. Reduction gearing is connected to the motor. The reduction gearing can be configured for reciprocating the magnet and thereby reciprocating the movable element. The reduction gearing can be configured for rotatably reciprocating the magnet and thereby rotatably reciprocating the movable element.

A liquid coagulation measuring device has a case and a reciprocating motor within the case. Reduction gearing can be connected to the motor. A contactless coupling can be connected to the reduction gearing and be configured for reciprocating a movable object in a well within the case. A temperature controller controls temperature within the case. A compact microscope in the case can be configured for magnifying an image of a movable object placed within the case.

A light source can illuminate the liquid or the movable object placed within the case. A video camera records movement of the movable object placed within the case. A power source can be connected to the motor, the light source and the video camera. A central processor can be connected to the power source and to the video camera and records a time from start of movement of the movable object until a change of the movement.

A display can be connected to the central processor. A smartphone connected to the case provides control of the light source, the video camera, the central processor and the display.

A rectangular box can include a bottom, a top and sides connecting the bottom and the top, supporting the smartphone on the top. A cover has a large opening with a frame for exposing the display and a start button of the smartphone while holding the smartphone on the box. An opening in at least one of the sides receives a cartridge having the liquid well. A pusher can be connected to the reduction gearing for pushing a lid on the cartridge and dropping the movable object into the well.

A measuring device can be turned on. Internal temperature can be controlled in the device. A cartridge can be inserted into the device beneath a small microscope or a magnifier. A liquid sample can be injected into a well within the cartridge. The well or a movable device therein is reciprocated. The movable device is illuminated and is observed through the microscope with a video camera. Times of changes in movement of the movable device can be recorded. The movable device can be reciprocated with a contactless magnetic coupling. Time differentiation is recorded between a start of movement of the movable device and slowing and stopping of movement of the movable device. The movable device can be placed in the well after the injecting of the liquid sample. A power source can be connected to the heat controller and the motor. The smartphone provides the illuminating and a video camera and a central processor for recording times of changes in movement of the movable device and creating displays according to the changes in movement of the movable device.

A smartphone connected to the measuring device can be turned on to start the illuminating, the video camera and the central processor.

Disclosed herein are a method for measuring coagulation of a liquid, comprising: turning on a measuring device, controlling internal temperature in the device, inserting a cartridge into the device, injecting a liquid sample into a well within the cartridge, the well comprising a sidewall and a floor, wherein the well floor is relatively hydrophilic, and the well wall is relatively hydrophobic with respect to the well floor; providing a contactless magnetic coupling, reciprocating the well or a movable device within the well with the magnetic coupling, illuminating the movable device, observing the movable device with a video camera, and recording times of changes in movement of the movable device.

In some configurations, the reciprocating comprises reciprocating the movable device with a contactless magnetic coupling, and the recording comprises recording time differentiation between a start of movement of the movable device and slowing and stopping of movement of the movable device.

In some configurations, the method further comprises placing the movable device in the well after the injecting of the liquid sample.

In some configurations, the turning on comprises connecting a power source to the heat controller and to a motor for the reciprocating, and starting the illuminating and a video camera and a central processor for recording times of changes in movement of the movable device and creating displays according to the changes in movement of the movable device.

In some configurations, starting the illuminating, the video camera and the central processor comprises turning on a smartphone connected to the measuring device.

Also disclosed herein is a method for measuring coagulation of a sample, comprising: activating a measuring device; inserting a cartridge into the measuring device; placing a liquid sample into a well within the cartridge, the well comprising a well wall and a well floor, the well further comprising a disc, the disc comprising a first tracking point comprising a first color, the first tracking point proximate a rotational center of the disc, the disc also comprising a second tracking point comprising a second color spaced apart from the rotational center of the disc, the first color different from the second color, wherein the well floor is relatively hydrophilic, and the well wall is relatively hydrophobic with respect to the well floor; activating a magnetic field of the measuring device; rotating the disc in a first direction using the magnetic field; rotating the disc in a second direction opposite the first direction using the magnetic field; illuminating the disc; tracking the first tracking point and the second tracking point of the disc with a video camera; and calculating changes in movement of the second tracking point with respect to the first tracking point of the disc with a processor to determine coagulation parameters.

In some configurations, calculating changes in movement of the first tracking point and the second tracking point of the disc occurs in real time.

In some configurations, the magnetic field comprises a contactless magnetic coupling.

In some configurations, the method comprises displaying the coagulation parameters on a display.

In some configurations, the display is a smartphone display.

In some configurations, rotating the disc in a first direction comprises rotating the disc 4°45′ degrees over 10 seconds.

In some configurations, the method comprises controlling an internal temperature in the measuring device.

In some configurations, the disc further comprises a spindle, such that the disc is spaced apart from a floor of the well.

In some configurations, the disc comprises ferromagnetic material to facilitate rotating the disc in the first direction using the magnetic field.

In some configurations, tracking comprises tracking a reduction in motion of the second tracking point with respect to the first tracking point as the magnetic field becomes no longer strong enough to overcome viscoelasticity of the liquid sample as the liquid sample coagulates.

In some embodiments, a method for measuring coagulation of a sample, comprising: activating a measuring device; inserting a cartridge into the measuring device; placing a liquid sample into a well within the cartridge, the well comprising a well wall and a well floor, the well further comprising a disc, the disc comprising a first tracking point comprising a first color, the first tracking point proximate a rotational center of the disc, the disc also comprising a second tracking point comprising a second color spaced apart from the rotational center of the disc, the first color different from the second color, the disc spaced apart from a well floor via a spindle operably connected to the disc, wherein the well floor is relatively hydrophilic, and the well wall is relatively hydrophobic with respect to the well floor; controlling an internal temperature of the measuring device; activating a magnetic field of the measuring device; rotating the disc in a first direction using the magnetic field; rotating the disc in a second direction opposite the first direction using the magnetic field; illuminating the disc; tracking the first tracking point and the second tracking point of the disc with a camera; and calculating changes in movement of the second tracking point with respect to the first tracking point of the disc with a processor to determine coagulation parameters. In some embodiments, tracking comprises tracking a reduction in motion of the second tracking point with respect to the first tracking point over time as the magnetic field becomes no longer strong enough to overcome viscoelasticity of the liquid sample as the liquid sample coagulates.

In some configurations, the well floor comprises a hydrophilic coating.

In some configurations, the well wall comprises a hydrophobic coating.

In some configurations, the disc is positioned substantially parallel to the well floor.

In some configurations, a disc diameter to well diameter can be between about 0.5 and about 1.0.

In some configurations, a disc diameter to well diameter can be between about 0.6 and about 0.8.

In some configurations, a well diameter to well depth ratio is between about 3.0 and about 6.0.

In some configurations, a well diameter to well depth ratio is between about 4.0 and about 5.0.

In some configurations, the method comprising validating the cartridge by observing indicia associated with the cartridge.

In some configurations, the indicia comprises a QR code or barcode.

In some configurations, the indicia comprises an RFID tag.

In some configurations, the indicia comprises cross-hairs.

In some embodiments, a cartridge for measuring coagulation of a sample, comprises any number of: a well configured to hold a liquid sample, the well comprising a well wall and a well floor, the well further comprising a disc, the disc comprising a first tracking point comprising a first color, the first tracking point proximate a rotational center of the disc, the disc also comprising a second tracking point comprising a second color spaced apart from the rotational center of the disc, the first color different from the second color, the disc spaced apart from a well floor via a spindle operably connected to the disc, wherein the well floor is relatively hydrophilic, and the well wall is relatively hydrophobic with respect to the well floor.

In some configurations, the well floor comprises a hydrophilic coating.

In some configurations, the well wall comprises a hydrophobic coating.

In some configurations, the disc is positioned substantially parallel to the well floor.

In some configurations, a disc diameter to well diameter can be between about 0.5 and about 1.0.

In some configurations, a disc diameter to well diameter can be between about 0.6 and about 0.8.

In some configurations, a well diameter to well depth ratio is between about 3.0 and about 6.0.

In some configurations, a well diameter to well depth ratio is between about 4.0 and about 5.0.

These and further and other objects and features of some embodiments of the invention are apparent in the disclosure, which includes the above and ongoing written specification, with the claims and the drawings.

### BRIEF DESCRIPTION OF THE DRAWINGS

FIG. 1A is a CAD rendering of the new Coagulation Profiler and subsystems.

FIG. 1B is a prototype showing blood placed on cartridge is loaded into the analysis slot.

FIG. 1C is a conceptual rendering of cartridge actuation and sub system.

FIG. 1D is a description of displayed profile, relative to cartridge actuation.

FIG. 1E is a detailed description of BioMEMS coagulation profile cartridge.

FIG. 1F is an image describing BioMEMS device marking relative to tracking.

FIG. 2A are images describing translation of device motion into zero amplitude profile.

FIG. 2B are images describing translation of device motion into max amplitude (MA) profile.

FIG. 3 shows an alternate embodiment with a fixed magnet.

FIG. 4 shows an alternate embodiment with torsion springs.

FIG. 5 is an image of a prototype showing accuracy of Level I control fluid.

FIG. 6 is an image of a prototype showing accuracy of Level II control fluid.

FIG. 7 is an image of a prototype showing repeatability of Level I profiles using two separate cartridges.

FIG. 8 is an SEM image showing fibrin adhesion into surface of HDDA.

FIG. 9 shows a forward surgical team attending a soldier.

FIGS. 10A and 10B show prior art TEG and ROTEM devices.

FIGS. 11A-15 show examples of cartridges.

FIGS. 11A and 11B are perspective and a schematic side view showing an example of a cartridge, platform, well, extended lid, an abutment on the platform and a disk attached to the lid, all of which are inserted in a receiver before fluid is injected into the well.

FIGS. 12A and 12B are perspective and a schematic side view showing a cartridge, platform, well, extended lid, an abutment on the platform and a disk attached to the lid. Moving the lid drops the disk into the well after fluid is injected into the well.

FIGS. 13A and 13B are perspective and a schematic side view showing a cartridge, platform, well, extended cover or lid, an abutment on the platform and a disk attached to the lid, all of which are inserted in a receiver before fluid is injected into the well. After fluid is injected, the lid is pushed back and the disk is dropped from the lid into the well.

FIG. 14 is a perspective view of a cartridge without a lid showing a fluid injection port at one end of a passageway to the well and a retaining clip for retaining the cartridge in the receiver.

FIG. 15 shows a two-piece injection construction of the cartridge platform for compatibility with manufacture by injection molding.

FIG. 16 shows loading protocols and the coagulation profiler with a disposable cartridge inserted and a blood sample ready to be loaded in a port in a portion of the cartridge extended from the case.

FIG. 17 shows a reciprocating motor with reduction gears and temperature controlling fins mounted on the motor housing.

FIG. 18 shows the reduction gearing and extended fins of the temperature controller near the cartridge and a pusher connected to the reduction gears for pushing a lid back onto the cartridge for dislodging and dropping the magnetized disk on blood in the cartridge well.

FIGS. 19-28 disclose additional embodiments relating to medical analyzers and removable cartridges.

### DETAILED DESCRIPTION

The invention provides, in some aspects, a handheld medical analyzer platform and biological microelectromechanical systems (BioMEMS) cartridges. This combined system uses microfluidics, optics, a mobile device (e.g. a smartphone or tablet) and video analysis software to create a handheld analyzer that produces data used in medical and biological diagnostics. In this embodiment, two primary components are the handheld medical analyzer and the coagulation profile cartridge. The combination of the handheld analyzer and coagulation profile cartridge can provide results equal to bench top systems used in hospitals, such as TEG and ROTEM. The handheld medical analyzer is a platform that is capable of analyzing a variety of cartridges. However, the coagulation profile cartridge may be specific to coagulopathy applications only.

Although the cartridges are intended to be disposable, they also can be implemented in a permanent fashion when cleaned properly and constructed of the proper material. Combined, the handheld medical analyzer and coagulation profile cartridge produce a coagulation profile which is displayed and stored on the analyzer. In some embodiments of the invention, the cartridge provides data used in diagnosing different forms of coagulopathy.

Although the combination of the handheld analyzer and coagulation profile cartridge is one part of the invention, the handheld analyzer is not limited to analyzing this specific cartridge.

Other similar embodiments include profiling the coagulation of Limulus amebocyte lysate (LAL) or other analytes. In this case the extent of LAL coagulation would be representative of the presence of gram negative bacteria, since the LAL reacts with bacterial endotoxin or lipopolysaccharide (LPS).

A similar cartridge would also apply to other assays that detect a physical change in the sample, such a viscosity, elasticity or viscoelasticity. Examples of these embodiments may include saliva, cervical mucus or other body fluids.

Furthermore the handheld analyzer is also capable of using the same basic configuration to analyze a great many cartridges. These embodiments could also capture data using the video camera and interrogated using the CPU and GPU running software. These cartridges include, but are not limited to CBC, HTC, PaO₂, pH and blood type.

Likewise similar use of a smartphone for cartridge analysis is not limited to video input, but also could use many other sensors on the smartphone, including direct electrical signals, wireless signals, manometers, accelerometers, gyroscopes and compasses. This includes combinations of the different methods of obtaining direct sensor information and indirect supplementary sensor information. An example of this would be using the combined system to provide a coagulation profile, while using the smartphone, wireless communication, accelerometers, gyroscopes, GPS, etc. to provide stabilization in rough environments such as a helicopter which is in motion and vibrating. These subsystems could also be used to send the coagulation profile, GPS coordinates to the ER providing an estimated time of arrival (ETA) and allowing for preparation of blood products, etc., in advanced for the patient's arrival.

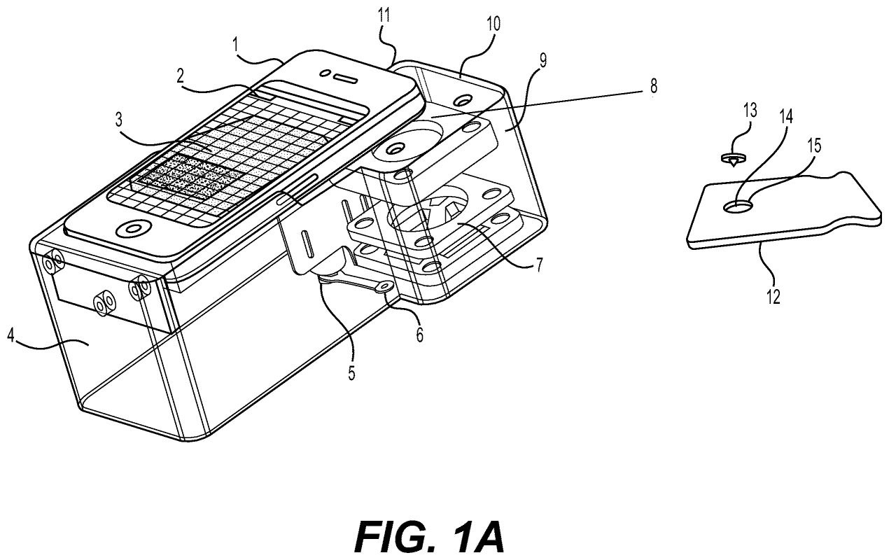

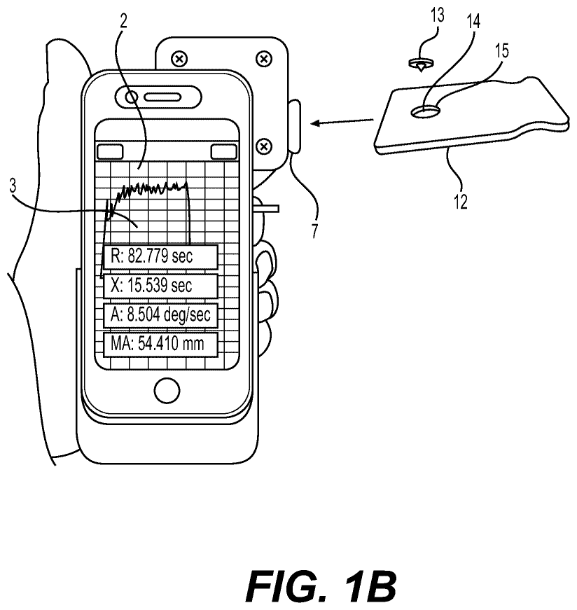

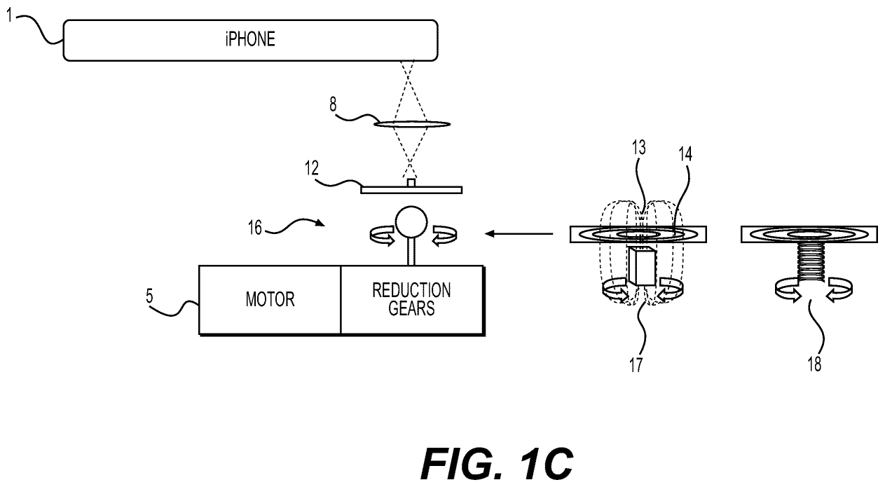

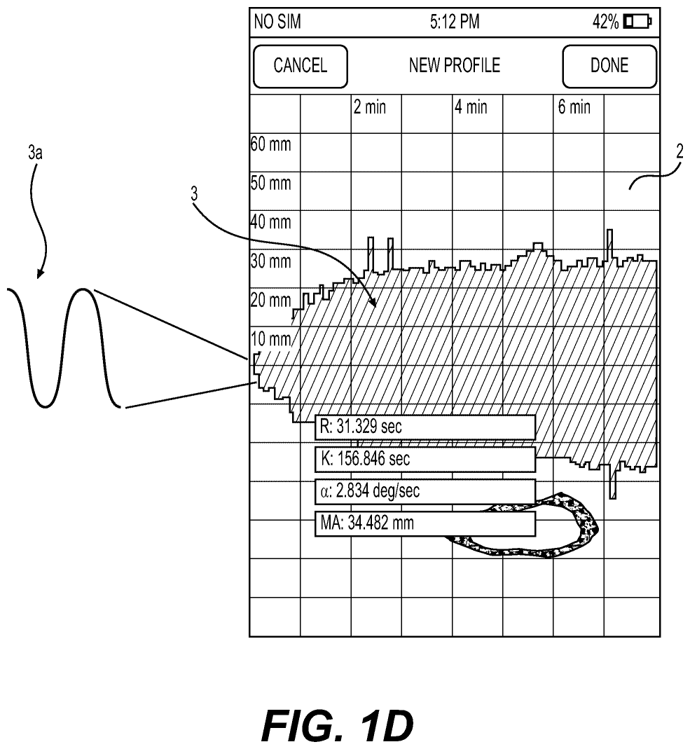

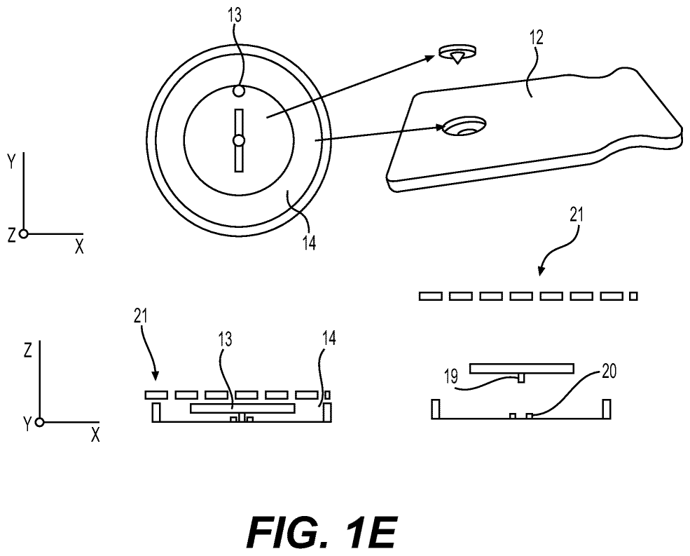

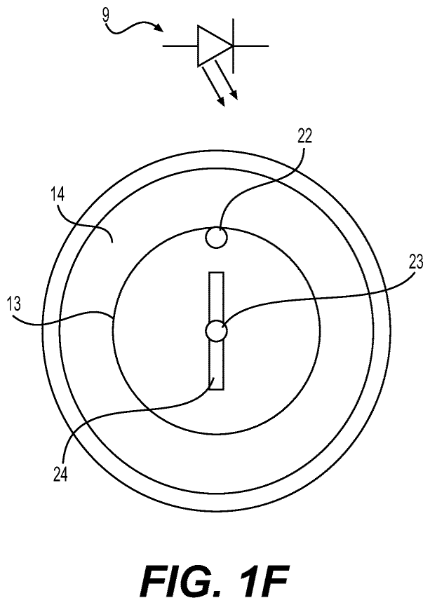

One embodiment is shown in FIG. 1A. The coagulation profile cartridge 12 is inserted into the cartridge slot 7 where the BioMEMS device motion interfaces with the sample 15 and the motion is captured using the smartphone 1 video camera 11 and is analyzed using the smartphone central processing unit (CPU), graphics processor unit (GPU) running proprietary software. The resultant coagulation profile 3 is displayed 2 on smartphone 1 (e.g. iPhone) screen along with the measured parameters, similar to TEG/ROTEM, Table 1, Table 2. A custom enclosure 4 provides a docking point for the smartphone and attachment of peripheral components. In this embodiment the peripheral components consist of a motor 5, mechanical linkage 6 and compact microscope assembly 8, light source 9 and temperature control unit 10. The motor and linkage can be replaced with a servo and gearing, to provide the desired rotational actuation.

The coagulation profile cartridge 12 is interrogated using the compact microscope 8 and video camera 11.

The loading protocol for the simplest embodiment of the combined system is: place blood 15 into well 14 on cartridge 12 and load the cartridge into analysis slot 7, also shown in FIG. 1B.

The disc 13 may be removed prior to filling the well 14, or the well may be filled with the disc in place. In this simple embodiment the well would be filled using a pipette.

Upon loading the cartridge the measurement begins as the disk is actuated, as shown in FIG. 1C. Actuation in this embodiment is performed using a motor 5, mechanical linkage 6 in the form of reduction gears and magnet 16, which couples motion to the cartridge disc 13. Other embodiments would include electromagnetic induction or direct mechanical drive via a spring.

By embedding ferrous metal into the cartridge disc 13, the magnetic field 17 couples the disc with the magnet. This coupling forms a link analogous to a torsion spring 18. Motion is thereby induced into the disc by rotating the magnet. In this embodiment the rotation is ±4°45′ degrees over 10 seconds. Other embodiments would include any number variations in the angular rotation over time.

In the embodiment the degree to which the motion is decoupled is representative of the displayed 2 profile 3, as shown in FIG. 1D. Both processed profiles 3 and preprocessed traces are sinusoidal waveforms, e.g. 3a, that represent the motion or decoupled motion. The sinusoidal waveform is acquired using the video camera and the CPU and GPU running proprietary software.

The alpha numeric displays, as one non-limiting example:
R: 31.329 sec
K: 156.846 sec
a: 2.834 deg/sec
MA: 34.482 mm

FIG. 1E shows a cartridge 12, an enlarged well top view, side cross section and an exploded side cross section. As shown in FIG. 1E, the cartridge has a well 14 and a disc 13. The disc is seated in the well via a spindle 19 and a bearing cup 20, at the bottom of the well. Both the well and the disc are sealed in the cartridge with a clear plastic lid 21. The lid can be hinged, sliding to allow sample insertion, or a microfluidic channel maybe used to load the sample into the well. This particular subcomponent of the cartridge may be realized in a number of different embodiments. These range from a simple hole in the lid to more complex automated microfluidics channels and chambers which meter the appropriate amount of blood or other sample, and/or reagent into the well. Likewise the insertion of the sample may range from manually using a pipette to the use of automated microfluidics.

The motion of the disc is captured by tracking two points overtime. FIG. 1F shows the pivot point 23 at the center of the disc 13 and a tracking point 22 at the outside edge of the disc. Each point is a unique color. In this embodiment the colors are florescent when illuminated using a UV LED light source 9. Both points are tracked by selecting the color to be tracked on the smartphone screen followed by selecting a threshold for the hues of the selected color to be included. The centroid of each of the prescribed points is then calculated to determine the exact location to be tracked. The angular motion is then calculated by comparing the centroid of the pivot point 23 to the centroid of the tracking point 22. This calculation can be performed real-time and is used to calculate the displayed profile continuously over time.

In some embodiments, color and shape can be used to track the tracking point and the pivot point. In some embodiments, an algorithm can utilize one, two, or more of hue, saturation, brightness, size, shape, convexity, circularity. This can be sufficient in most cases, however there are multiple variations of the orientation of the disk in the well and as the disk moves, these parameters sometimes change throughout the motion. Likewise there are sometimes reflections from the LEDs that can cause it to lose track. In some embodiments, reducing the reflections and color variation can be achieved by using UV LEDs that create a better contrast between the tracking points and the background. Likewise, decals that have a black background can be used further increasing the contrast between the tracking points and the background. Furthermore, machine vision tracking techniques can be incorporated to include machine learning.

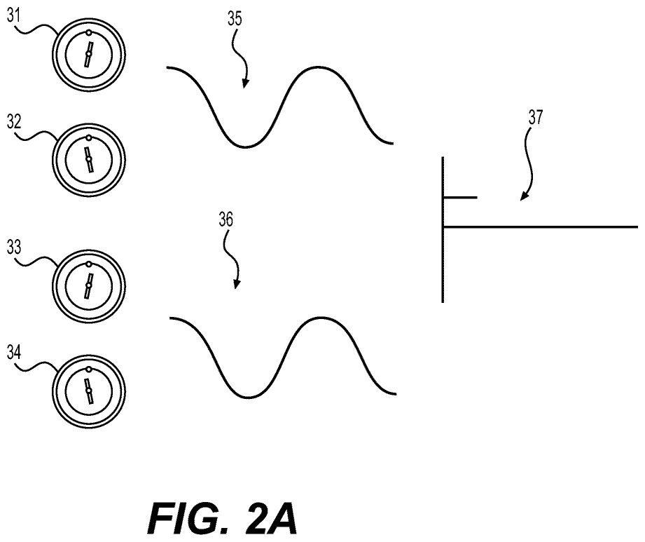

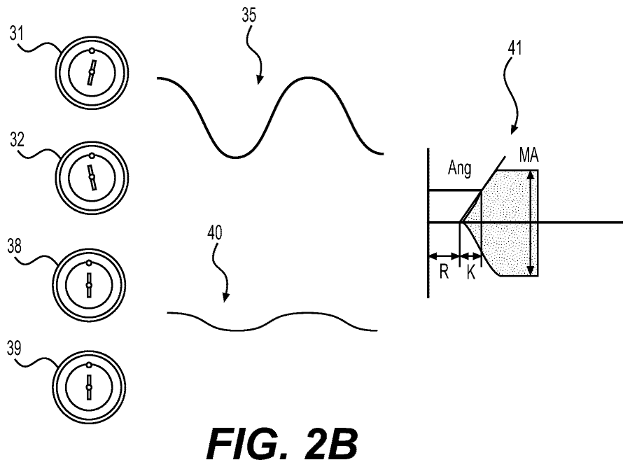

The detailed translation of the device motion is shown in FIG. 2A. At the beginning of the measurement the disc motion is uninhibited and rotates the full range of motion, plus 4°45′ 31 and minus 4°45′ 32, in the fluid tested. This initial motion, before the onset of coagulation, is recorded as the baseline trace 35. The baseline trace is then differenced with the subsequent motion trace 36 to detect coagulation, which inhibits the coupling of motion of the magnet with the motion of the disc. In this embodiment, the displayed trace 37 (coagulation profile) is the difference between the baseline trace 35 and the subsequent motion trace 36. Prior to coagulation the baseline trace 35 is the same as the pre coagulation subsequent motion trace 36, and the coagulation profile 37 is the difference between the two: zero. As the blood coagulates, the induced motion is decoupled, and the magnetic field is no longer strong enough to overcome the viscoelasticity of the blood, as shown in FIG. 2B. Initially the motion is uninhibited 31, 32 producing the baseline motion 35. When coagulation occurs, decoupling reduces the induced motion 38, 39. Upon coagulation the large baseline trace 35 is differenced with the small post coagulation subsequent motion trace 40, resulting in a large amplitude profile 41. The moment in time shown, is at maximum amplitude represented by the parameter similar to the TEG parameter MA. As shown at the right of FIG. 2B and in FIG. 1B, the “reaction time” is the calculated time in decimal fractions of a second for a clot to reach 2 mm, “22 mm time” is the time in decimal fractions of a second for a clot to reach 22 mm, and “angle” is the slope of the angle between “reaction time” and 22 mm time. The “max clot” is maximum strength of a clot indicated in mm.

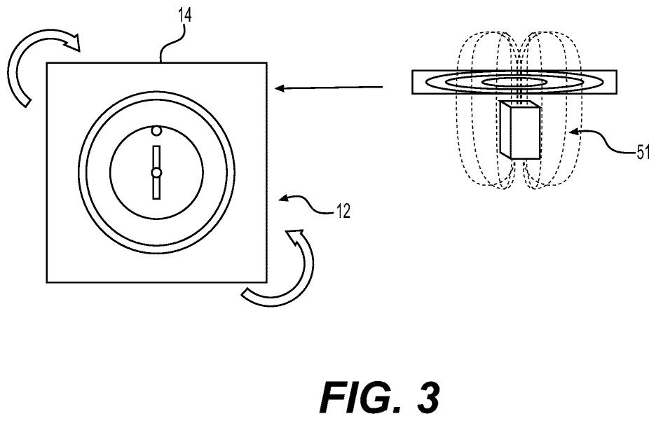

A second embodiment of the BioMEMS device is shown in FIG. 3. In this embodiment a fixed magnet 51 is coupled to the disc. In this fixed magnet case, the rotation would be induced by rotating the cartridge 12 or well 14 within the cartridge. In this fixed magnet embodiment, the motion induced to the disc would be traced directly, with no differencing necessary. In this case the tracked motion could be directly used as the profile trace. Prior to the onset of coagulation there would be no motion coupled and the disc would oscillate uninhibited within the well. With no motion induced, the coagulation profile would be zero. In the fixed magnet case as the coagulation increases, the coupling would increase inducing more motion as coagulation continues. At MA the maximum amount of motion would be induced through the magnet.

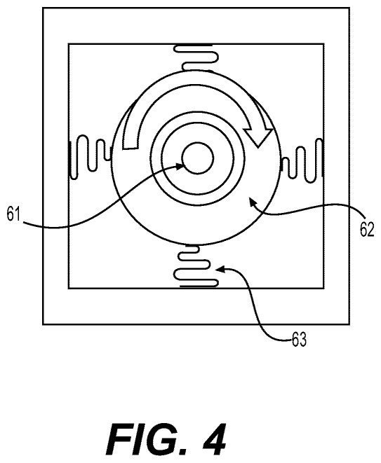

A third embodiment of a coagulation profiling BioMEMS device is shown FIG. 4. This device has a center disc 61 and an outer ring 62 that is suspended by torsion springs 63. As the blood begins to coagulate, the rotating inner disc 61, actuated via an oscillating magnet, couples to the outer ring 62. This couples a reciprocating motion to the outer ring 2. At MA the maximum amount of motion would be induced through the magnet.

The BioMEMS embodiments shown are not all of the possible variations. For instance, one embodiment could use a disc fixed to the center of the well and actuate a ferrous ring in the well.

The measurement provided by some embodiments of the invention is impervious to motion. Due to the extremely small dimensions of the BioMEMS device in some cases, compared to the conventional size of TEG and ROTEM, the measurement is highly impervious to motion. The small mass of the device and small volume residing in the well present less inertia when external motion is applied. The ability to produce a noise-free measurement in the presence of motion is further enhanced by the magnetic coupling, which fixes the disc and the well in the magnetic field.

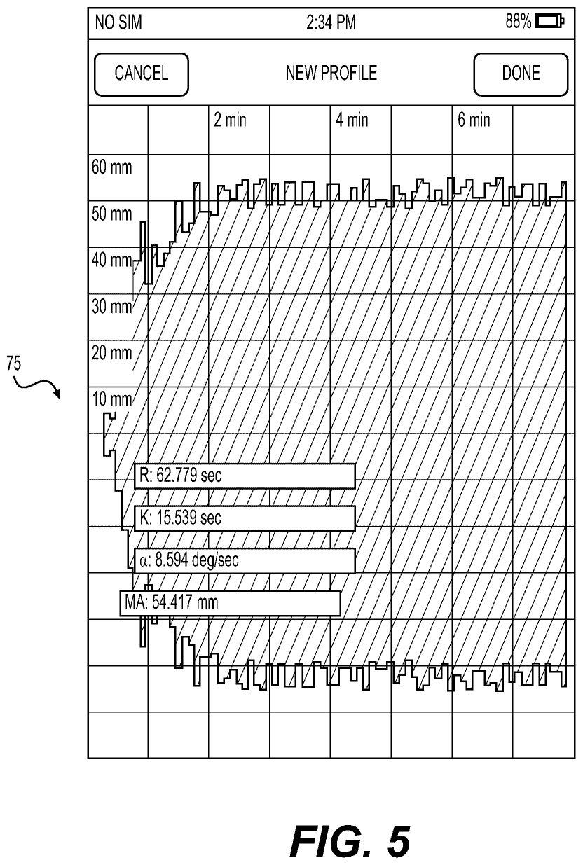

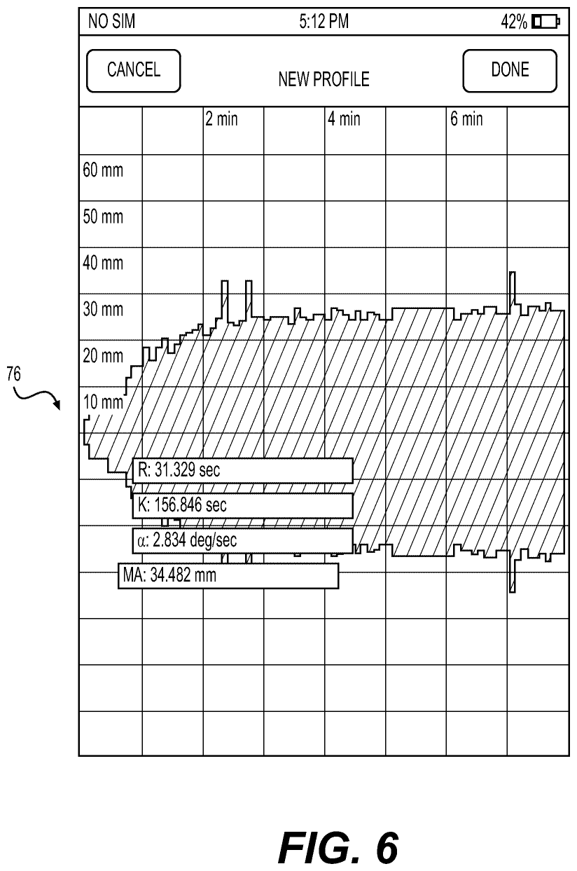

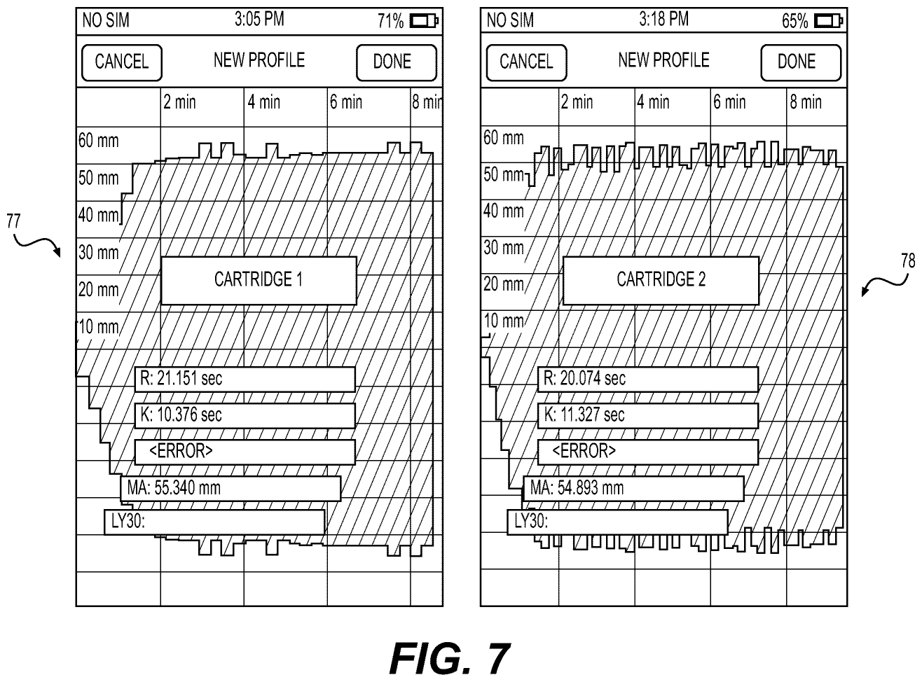

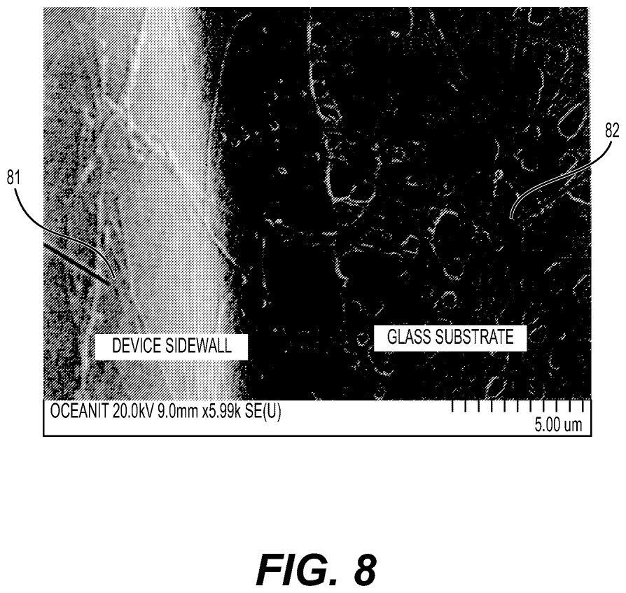

A prototype of an embodiment of the invention has provided concept validation. The image shown in FIG. 1B is an actual working prototype. The coagulation profile on the screen was taken using the prototype and shows the coagulation profile of using a quality control standard used and provided by Haemoscope, the makers of the TEG. A prototype of some embodiments of the invention has accurately differentiated between the Haemoscope Level 1 control (normal profile) as shown in screen 75 in FIG. 5 and the Level II control (abnormal profile) as shown on screen in FIG. 6. In addition repeatability has been demonstrated with multiple cartridges accurately measuring the samples with nearly identical results, as shown on screens 77 and 78 in FIG. 7. The polymer selection provides improved fibrin adhesion. The use of polymers for the fabrication of the cartridges has also been demonstrated to work well. In addition to being disposable and inexpensive to manufacture, the polymers have demonstrated advantages for us in some embodiments of this invention. Specifically, the use of HDDA promotes fibrinogen to be embedded into the polymer surface prior to the formation of fibrinogen. As the fibrinogen polymerizes it forms an excellent bond to the surfaces. This provides an ideal surface for detecting the viscoelasticity of the coagulating blood between the two HDDA surfaces. FIG. 8 shows fibrin 81 embedded into the surface of a HDDA disc sidewall. FIG. 8 also shows fibrin on the surface of the glass substrate, where air pockets are forming due to a lack of adhesion with no apparent fibrin embedded into the glass surface.

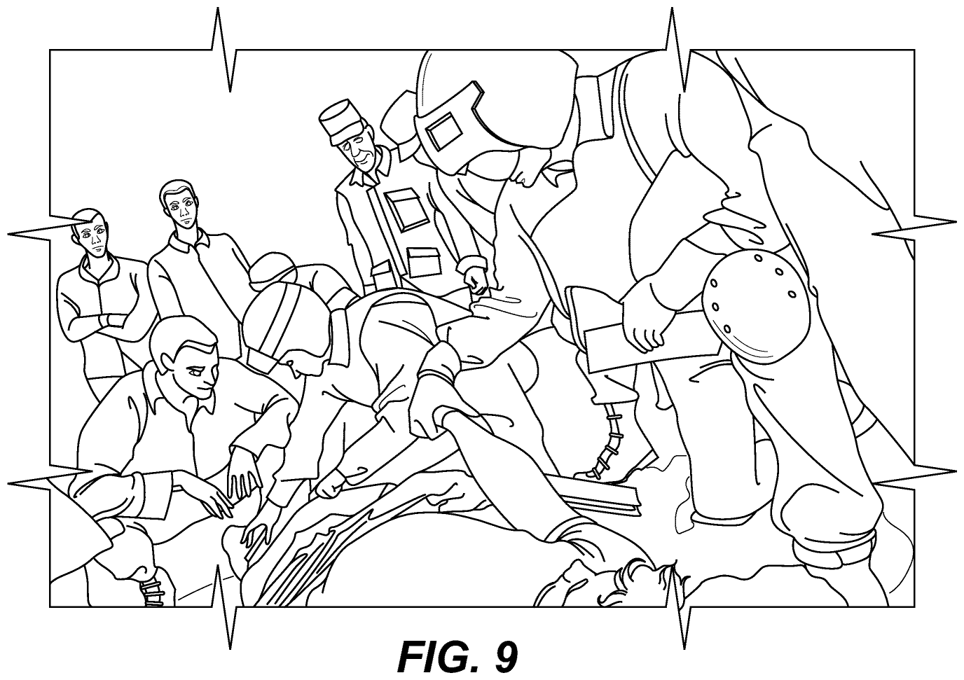

FIG. 9 shows a forward surgical team attending a soldier at a forward position where some embodiments of the present invention is needed, a similar case would be an EMT or paramedic attending a trauma victim.

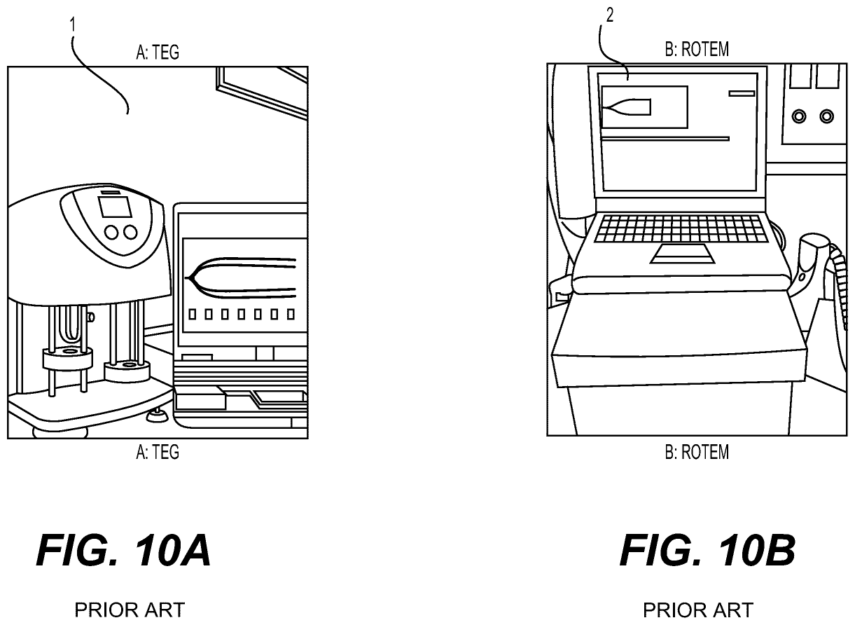

FIGS. 10A and 10B show prior art TEG and ROTEM devices.

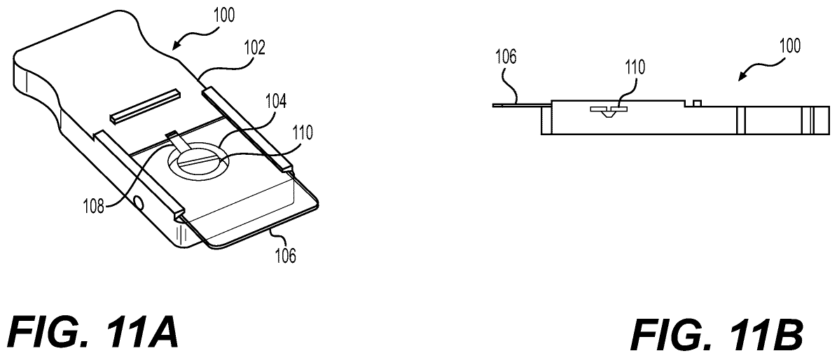

FIGS. 11A and 11B are perspective and a schematic side view showing additional features of the cartridge 100, a platform 102, a well 104, an extended lid 106, an abutment 108 on the platform and a disk 110 attached to the lid 108, all of which are inserted in a receiver before fluid is injected into the well 104. FIG. 11B is a schematic representation of the well 104 and the disk 110 which is attached to a bottom of the lid 106.

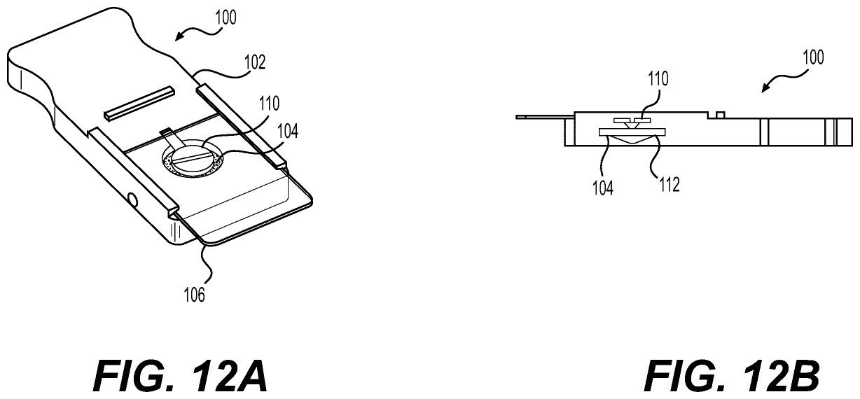

FIGS. 12A and 12B are perspective and a schematic side view showing disk loading technique of the cartridge 100, platform 102, the well 104, the extended lid 106, and an abutment 108 on the platform. Disk 110 is still attached to the lid 106.

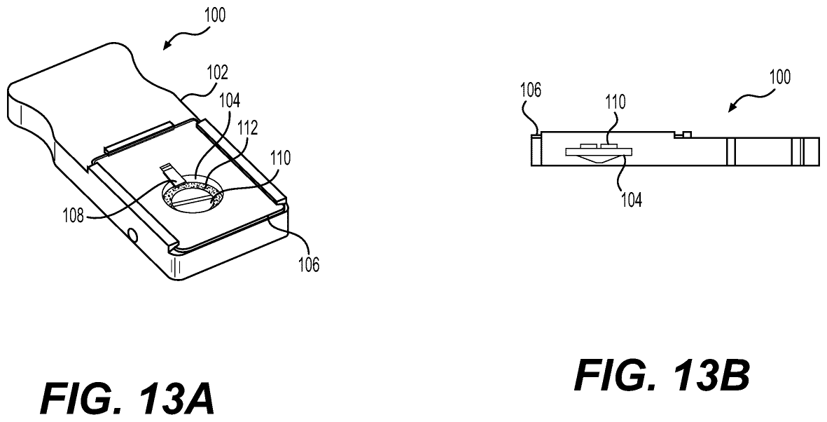

FIGS. 13A and 13B are perspective and a schematic side view showing a cartridge 100, the platform 102, well 104 and lid 106. An abutment 108 on the platform 102 has dislodged disk 110 from the lid after fluid is injected into the well and after lid 106 is pushed back and disk 110 is dislodged and dropped from the lid into the well.

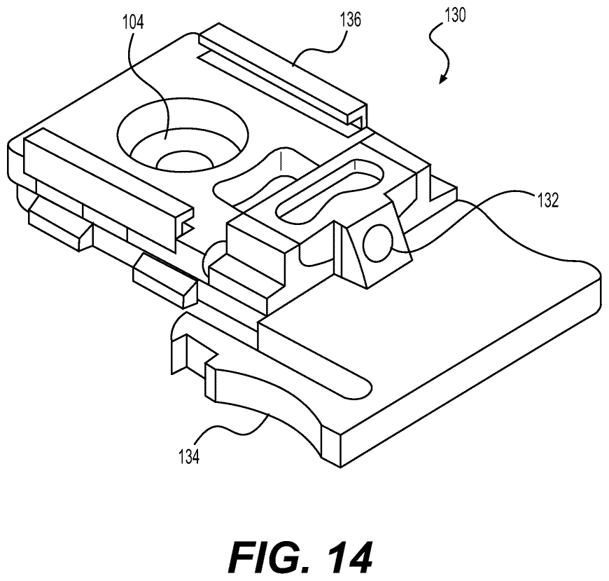

FIG. 14 is a perspective view of a cartridge 130, which shows additional features of an injection port and a retaining clip. In this case it is shown without a lid, showing a fluid injection port 132 at one end of a passageway to the well and retaining clip 134 for retaining the cartridge in the receiver. Upward and inward extending opposite guide rails 136 hold a lid 106, such as shown in FIGS. 11A-13B.

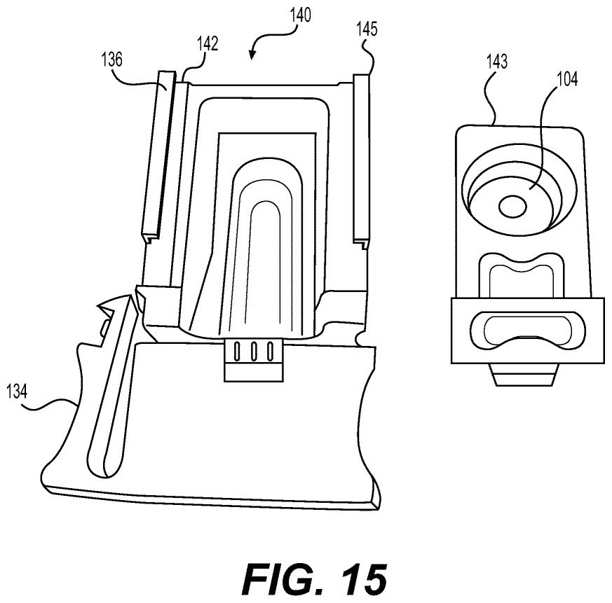

FIG. 15 shows a two-piece injection construction of a cartridge 140 platform 142 for compatibility with manufacture by injection molding. Inner part 143 holds the well 104, and outer part 145 has the retaining clip 134 and the guide rails 136.

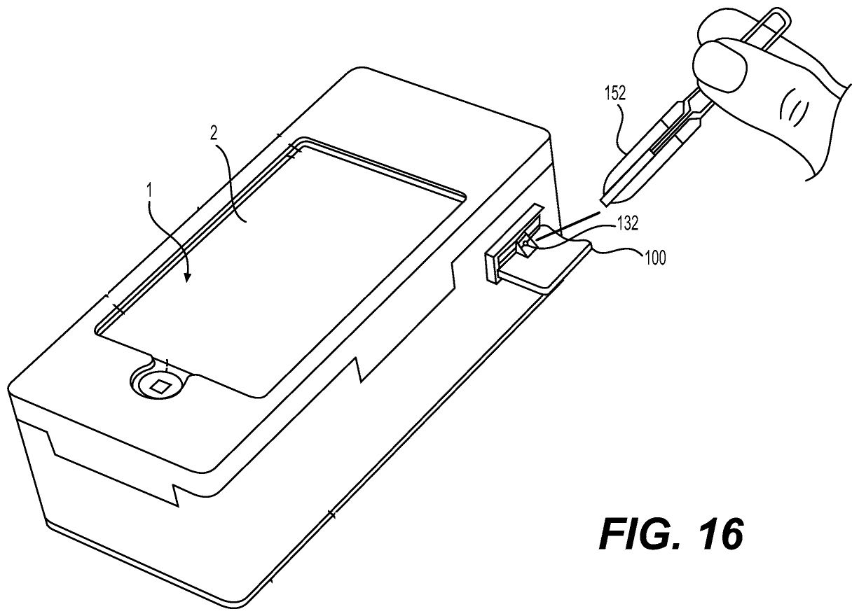

FIG. 16 shows loading protocols and the coagulation profiler with a disposable cartridge 100 inserted and a blood sample 152 ready to be loaded in a port 132 in a portion of the cartridge extended from the case. Also shown are smartphone 1 with display 2.

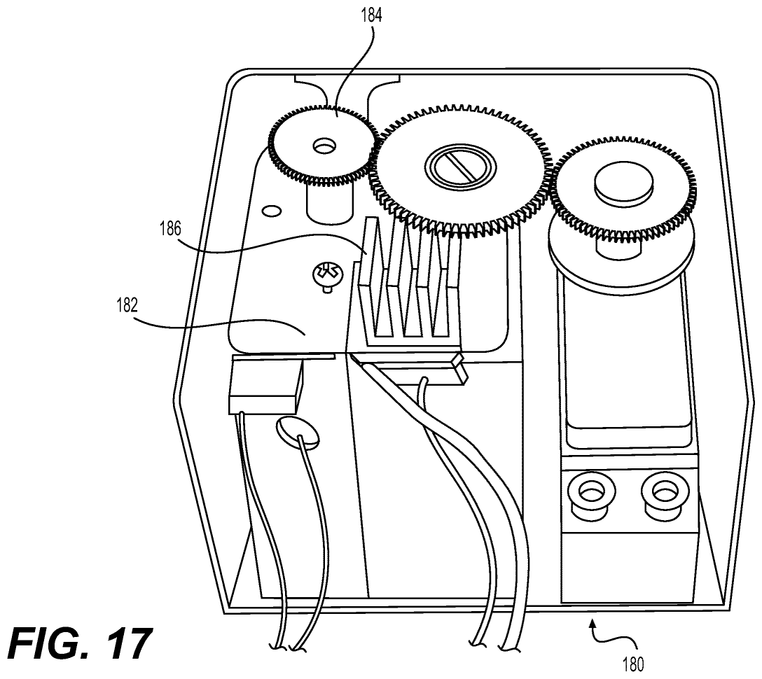

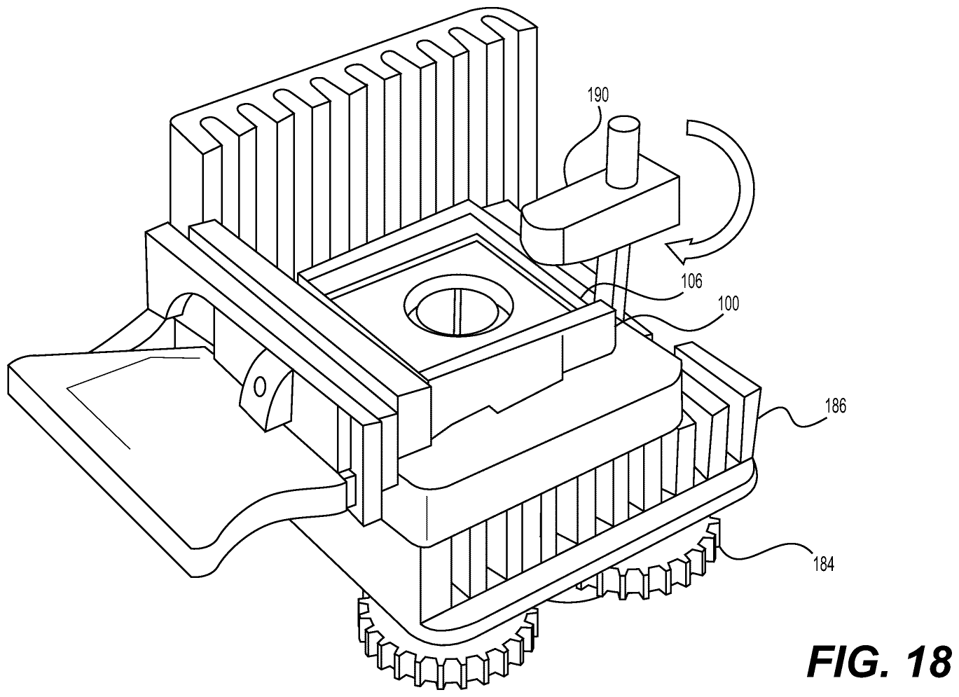

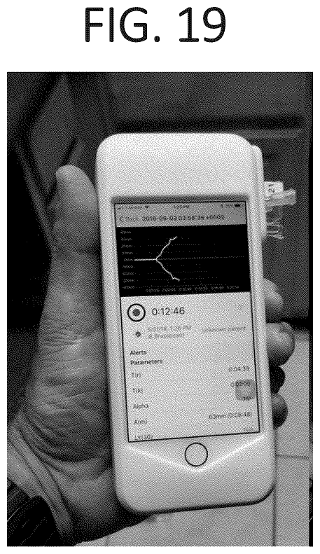

FIG. 17 shows a reciprocating motor 180 with reduction gears 184 and temperature controlling fins 186 mounted on the motor housing 182.

FIG. 18 shows the reduction gearing 184 and extended fins 186 of the temperature controller near the cartridge 100 and a pusher 190 connected to the reduction gears for pushing a lid 106 back onto the cartridge 100 for dislodging and dropping the magnetized disk on blood in the cartridge well.

FIG. 19 illustrates a non-limiting example of a smartphone interface for a medical analyzer.

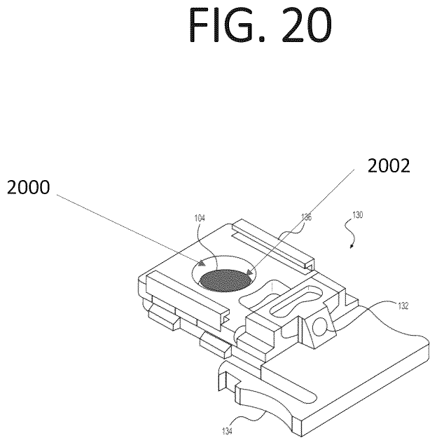

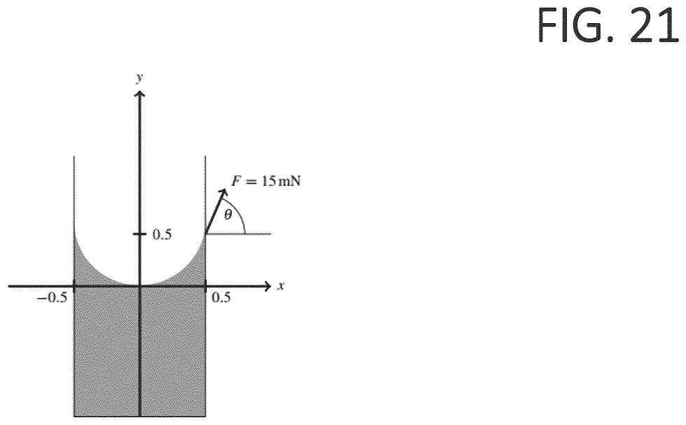

FIG. 20 schematically illustrates features of a cartridge similar to that described and illustrated in connection with FIG. 14, with a well including a well wall 2000 (sidewall) and a well floor 2002 including a perimeter defining a base of the well sidewall 2000. In some embodiments, the well floor 2002 can be relatively hydrophilic, while the well wall 2000 can be relatively hydrophobic.

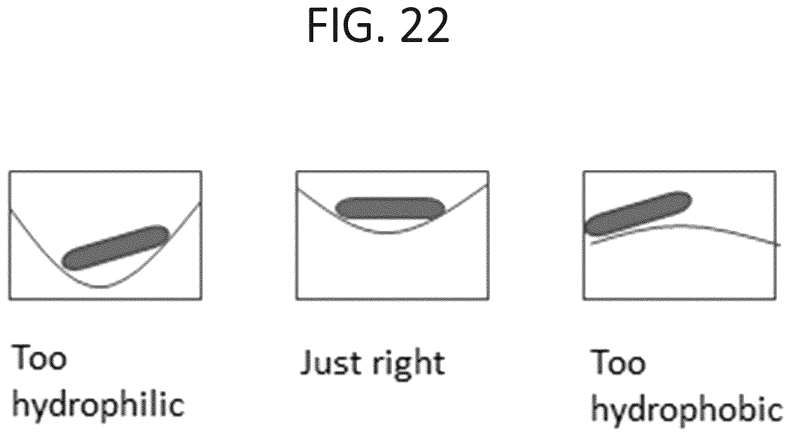

Liquid can form a meniscus, defined as the curve in the upper surface of a liquid close to the surface of the container or another object, caused by surface tension. It can be either concave or convex, depending on the liquid and the surface. A concave meniscus occurs when the particles of the liquid are more strongly attracted to the container (adhesion) than to each other (cohesion), causing the liquid to climb the walls of the container. This occurs between, for example, water and glass. Water-based fluids also have a concave meniscus in glass or other wettable containers. Conversely, a convex meniscus occurs when the particles in the liquid have a stronger attraction to each other than to the material of the container. Convex menisci occur, for example, between mercury and glass in barometers and thermometers. Further details regarding equations relating to meniscus dimensions and other properties that can be controlled according to a desired clinical result are described in FIG. 21. FIG. 22 illustrates schematically a disc positionable with respect to the meniscus of a fluid sample. The well wall and/or floor can be made or coated with materials having controllable properties with respect to water (e.g., relatively hydrophilic or hydrophobic) depending on the desired clinical result. As illustrated, in some embodiments, the well floor can be relatively hydrophilic, while the well wall can be relatively hydrophobic with respect to the well floor, resulting in a disc that can be acceptably be positioned substantially parallel to the well floor (center illustration), and otherwise angled with respect to the well floor if the well wall and/or floor are too hydrophobic (right illustration) or hydrophilic (left illustration). In some embodiments, the well wall and/or the well floor are configured such that the longitudinal axis of the disc is parallel to, or angled to within about 20, 15, 10, 9, 8, 7, 6, 5, 4, 3, 2, 1, or less degrees with respect to the longitudinal axis of the well floor.

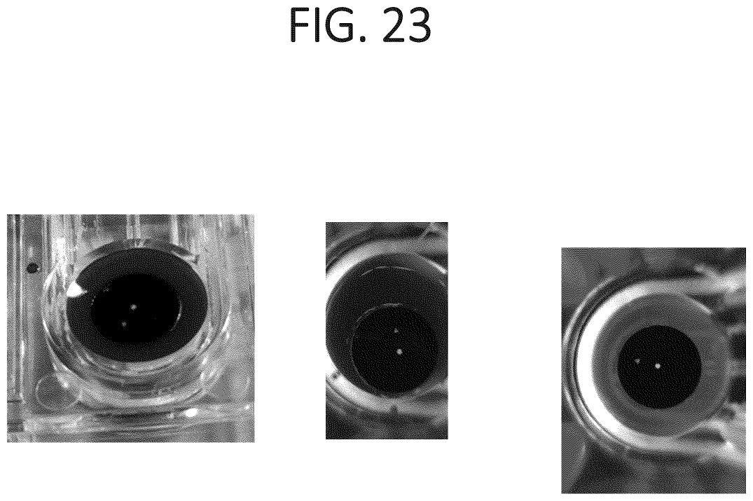

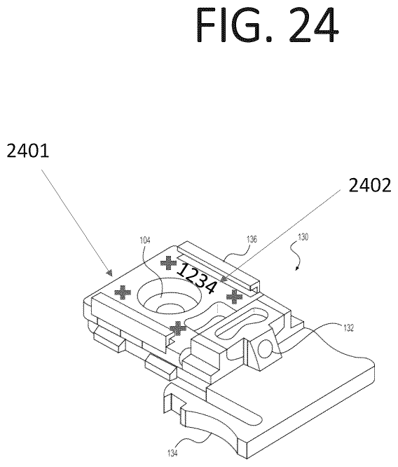

FIG. 23 schematically illustrates discs positioned within a well of a clear (relatively transparent cartridge) and illustrating proper central positioning of the disc (left and right drawings) and offset positioning of the disc (center) relating to a well wall and/or floor that is too hydrophilic and/or hydrophobic, respectively.

In some embodiments, a specific ratio of well wall and/or floor can be created to form a meniscus of particular dimensions. Also, a specific ratio of disc to well size can allow for creation of automatic positioning of the disc.

In some embodiments, the hydrophobicity or hydrophilicity of a well wall and/or floor can be controlled using a coating. In some embodiments, the hydrophobicity or hydrophilicity of a well wall and/or floor can be controlled using surface texturing features such as roughening, sintering, laser, chemical, or other etching, and the like. When the proper hydrophobic coating is applied to the well wall, the optimal meniscus is achieved at a defined volume. As the volume increases and approaches the lip of well, it becomes less concave and eventually becomes convex as it reaches the lip of the well. Therefore, in some cases the disk will only be centered in the well when the optimum volume is reached. By tracking the location of the disk as fluid is introduced, the volume of the well is indirectly measured. This relationship can be used to automate the injection of the fluid sample.

In some embodiments, the diameter of the disk is optimized for the particular meniscus and desired fluid volume. In this case the ratio of disk diameter to well diameter and well depth can be determined.

In some embodiments, the disk diameter can be, for example, between about 3 mm and about 15 mm, between about 5 mm and about 9 mm, between about 6 mm and about 8 mm, or about, at least about, or no more than about 2, 3, 4, 5, 6, 7, 8, 9, 10, 11, 12, 13, 14, 15, 16, 17, 18, 19, 20 mm, or more or less, or ranges including any two of the foregoing values.

In some embodiments, the well diameter (e.g., at the top or bottom of the well) can be, for example, between about 4 mm and about 25 mm, between about 6 mm and about 15 mm, between about 8 mm and about 12 mm, or about, at least about, or no more than about 3, 4, 5, 6, 7, 8, 9, 10, 11, 12, 13, 14, 15, 16, 17, 18, 19, 20, 21, 22, 23, 24, 25 mm, or more or less, or ranges including any two of the foregoing values.

In some embodiments, the well depth can be, for example, between about 0.5 mm and about 8 mm, between about 1 mm and about 6 mm, between about 1.5 mm and about 3 mm, or about, at least about, or no more than about 0.5, 1, 1.5, 2, 2.5, 3, 3.5, 4, 4.5, 5, 5.5, 6, 6.5, 7, 7.5, 8 mm, or more or less, or ranges including any two of the foregoing values.

In some embodiments, the disk diameter to well diameter ratio can be, for example, between about 0.5 and about 1.0, between about 0.6 and about 0.8, or about 0.3, 0.4, 0.5, 0.6, 0.7, 0.8, 0.9, 1.0, or ranges including any two of the foregoing values.

In some embodiments, the well diameter to well depth ratio can be, for example, between about 3.0 and about 6.0, between about 4.0 and about 5.0, or about 2.5, 3.0, 3.5, 4.0, 4.5, 5.0, 5.5, 6.0, 6.5, 7.0, 7.5, 8.0, or ranges including any two of the foregoing values.

In some embodiments, the hydrophobicity is optimized for blood, however other fluids (e.g., urine, saliva, or other body fluids) may require variations in the hydrophobicity to achieve the desired meniscus.

One embodiment of the induced disc rotation would progressively reduce the range of motion to improve sensitivity. For instance, at the beginning of the test the motion induced would be +/−20 degrees. Since the range of motion is larger, smaller amounts of change are more easily detected and since the fibrin and platelets have yet to form a clot, the excess motion will not destroy the clot.

In an alternate embodiment an absorbent coating or material can be used to wick the fluid across the well floor. This provides even fluid distribution while maintaining hydrophobic walls.

In an alternate embodiment multiple perforation across the well floor provide multiple points of fluid entry. This could be a few points or a large number where the well floor is essentially a mesh. This provides even fluid distribution while maintaining hydrophobic walls.

FIG. 24 schematically illustrates an embodiment of a removable and/or insertable cartridge that can include indicia 2401, such as cross-hairs to ensure that the cartridge is fully inserted into the system. A QR code, bar code, serial number, RFID chip, or similar 2402 could be printed or otherwise attached to prevent reuse and counterfeiting. In some embodiments, a holographic image could be utilized to detect distance via color or other parameters. In some embodiments, exposure to UV light could remove the QR code or similar indicia, rendering the cartridge unstable and/or marking the cartridge as being used, thus preventing reuse.

In some embodiments, the cartridge or a portion thereof could be clear, optically transparent, or optically translucent to allow light to illuminate the well and allow the viewing of the flow of the blood or other sample into the cartridge. The cartridge could include a portion that is optically opaque (e.g., a blacked-out handle) for grip, blocking light from flowing through the clear material (e.g., plastic) and sealing the gap between the cartridge and the slot.

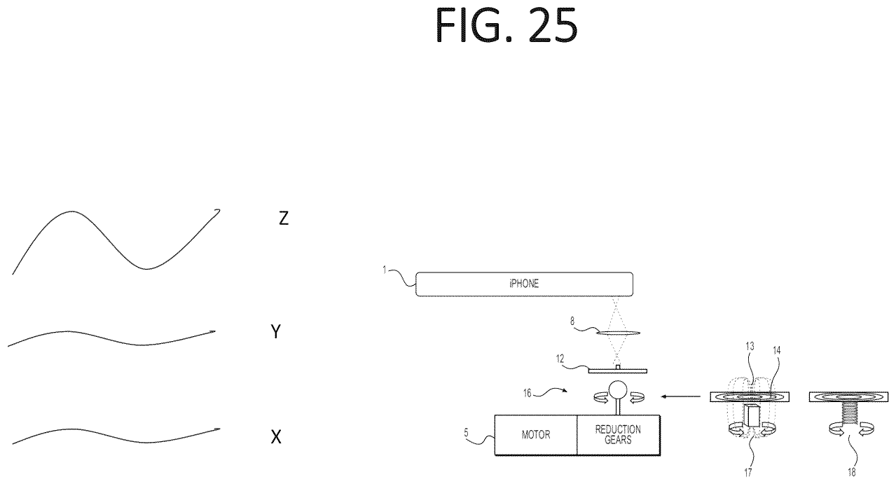

In some embodiments, such as shown in FIG. 25, calibrations can be performed to better ensure more accurate distance placement (proximity) between the cartridge containing the disk and the magnet which induces motion. Since the location of the magnetometer is fixed in the system, the relative magnetic field measured is representative of the components, e.g., three components being in the proper alignment. Likewise the actuation can be measured since it will change the magnetic field over time. Therefore if a sinusoidal waveform is of sufficient amplitude and frequency, the magnet can be the proper strength, in the proper location and oscillating over the proper range. The disc can be positioned within the cartridge when inserted into the analyzer system. A sensor such as a magnetometer within the imaging device (e.g., smartphone camera for example) or elsewhere could be utilized and calibrations can be performed. Example traces X, Y, and Z are shown, with trace Z illustrating a higher amplitude and potentially proper proximity, while traces X and Y are lower amplitude which may indicate a troubleshooting issue, e.g., improper proximity. Calibration and/or the magnetic field can lead to better clinical results, and an acceptable trace as illustrated and described previously in connection with FIG. 2A above.

In some embodiments, it can be advantageous to include a tracking point impervious to motion, as illustrated and described in connection with FIG. 1F. Localized tracking and reference point(s) can be co-located making it impervious to induced motion.

In some embodiments, the disc material density is between about 1.0 and about 1.5 g/cm³, such as between about 1.10 and about 1.30 g/cm³, between about 1.20-1.22 g/cm³, about 1.15, 1.16, 1.17, 1.18, 1.19, 1.20, 1.21, 1.22, 1.23, 1.24, 1.25 g/cm³, or ranges incorporating any two of the aforementioned values. In some embodiments, the permeability of the disk is zero or substantially zero. In some embodiments, an insert should extend partially or completely along the diameter or substantially the diameter of the disk with a ratio of about 15/1, 20/1, 25/1, 30/1, or ranges incorporating any of the foregoing values, and relative to the diameter of the disc. In some embodiments, the insert can have a permeability of between about 1×10⁻⁴ d or m² and about 6.3×10⁻³ d or m², between about 1×10⁻⁴ d or m² and about 6.3×10⁻³ d or m² or less than about 6.3×10⁻³ m² in some embodiments.

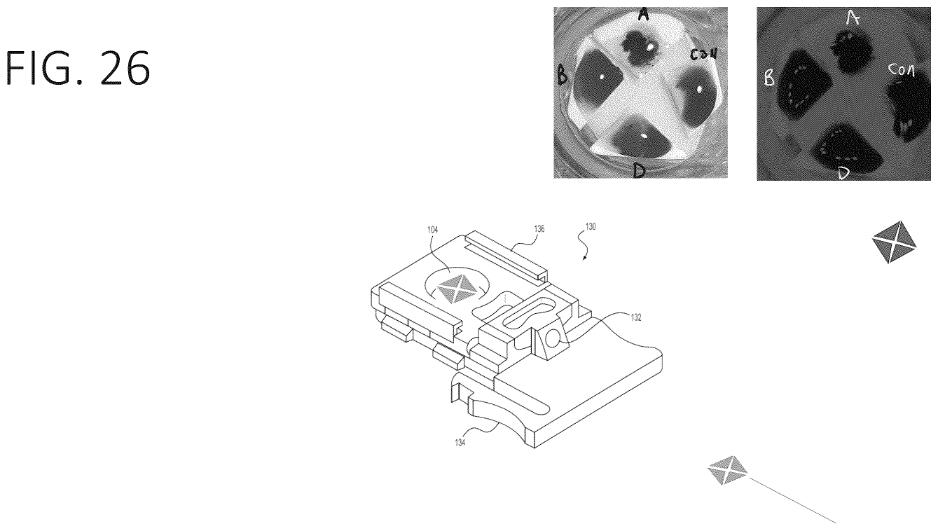

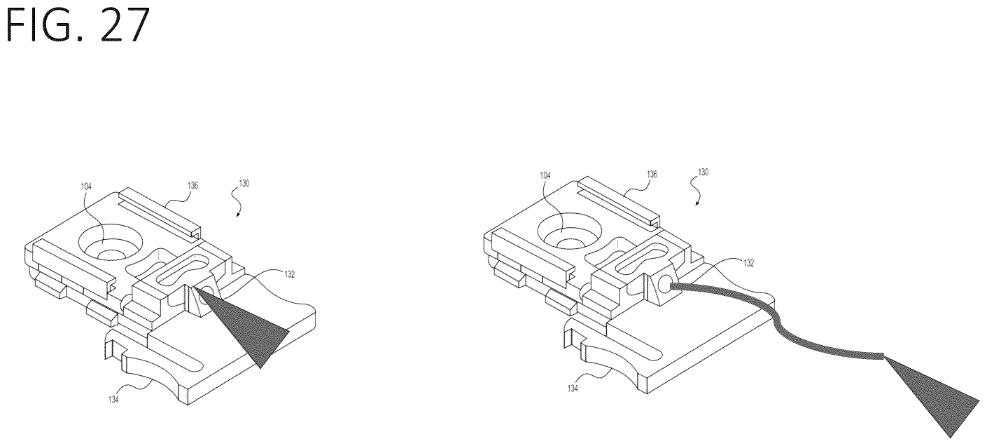

FIG. 26 illustrates an embodiment of a sample analysis system including an insertable cartridge that can be utilized for blood typing. In some embodiments, a plurality, such as 3, 4, or more separate compartments can be placed in a single well, each compartment having one, two or more reagents therein (e.g., 4 cartridges with 4 reagents; 1 reagent per cartridge). Each compartment or sub-well can include its own branch of an injection port, and have a defined shape, such as a triangular shape as shown, or other shapes (e.g., square, rectangular, rhomboid, trapezoidal, oval, circular, and others). A convex meniscus can provide a spherical shape to pull agglutination into the center of the mass. The triangular shape of hydrophobic well sections can remain a triangle of a blood-water (or other fluid/reagent) mixture. When agglutination occurs (positive typing), a circular shape can form as noted above. If no agglutination is present (negative typing), the well section including the sample can remain a triangular shape.

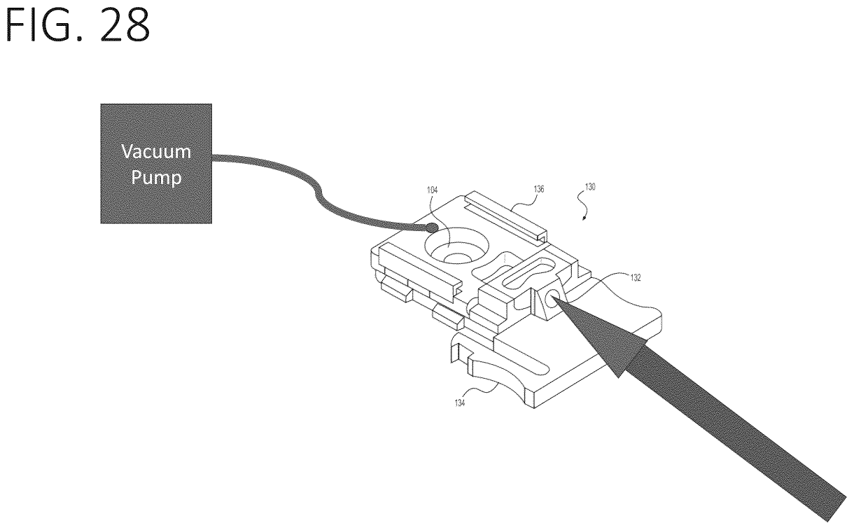

FIG. 27 schematically illustrates an embodiment of an insertable cartridge that can include a luer lock (at arrow apex) or other port (left figure) for connecting a syringe containing a sample. In some embodiments, the cartridge could be connectable to a tube (right figure) with a luer lock or other port spaced apart from the cartridge as shown.

FIG. 28 schematically illustrates an embodiment in which an insertable cartridge can include a sealed cartridge vacuum port. A vacuum pump can be in fluid communication with the sample well to allow a sample to flow into the well from the input port of the cartridge.

In some embodiments, the cartridge could be configured for a wide variety of applications, including but not limited to a catalase reaction, coagulase reaction, quick crossmatch, LAL detection, PT/INR, ACT, PTT, and the like. In some embodiments, a cartridge could include concentric wells configured for platelet mapping.

The foregoing description and examples has been set forth to illustrate the disclosure according to various embodiments and are not intended as being unduly limiting. The headings provided herein are for organizational purposes only and should not be used to limit embodiments. Each of the disclosed aspects and examples of the present disclosure may be considered individually or in combination with other aspects, examples, and variations of the disclosure. In addition, unless otherwise specified, none of the steps of the methods of the present disclosure are confined to any particular order of performance. References cited herein are incorporated by reference in their entirety.

While the methods and devices described herein may be susceptible to various modifications and alternative forms, specific examples thereof have been shown in the drawings and are herein described in detail. It should be understood, however, that the embodiments disclosed should cover modifications, equivalents, and alternatives falling within the spirit and scope of the various embodiments described herein and the appended claims.

Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some examples, acts or events can be performed concurrently, e.g., through multi-threaded processing, interrupt processing, or multiple processors or processor cores or on other parallel architectures, rather than sequentially.

The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed.

The various illustrative logical blocks, modules, processes, methods, and algorithms described in connection with the embodiments disclosed herein can be implemented as electronic hardware, computer software, or combinations of both. To clearly illustrate this interchangeability of hardware and software, various illustrative components, blocks, modules, operations, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration.

The blocks, operations, or steps of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, an optical disc (e.g., CD-ROM or DVD), or any other form of volatile or non-volatile computer-readable storage medium known in the art. A storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that some examples include, while other examples do not include, certain features, elements, and/or states. Thus, such conditional language is not generally intended to imply that features, elements, blocks, and/or states are in any way required for one or more examples or that one or more examples necessarily include logic for deciding, with or without author input or prompting, whether these features, elements and/or states are included or are to be performed in any particular embodiment.

The methods disclosed herein may include certain actions taken by a practitioner; however, the methods can also include any third-party instruction of those actions, either expressly or by implication. For example, actions such as “positioning a disc within a sample well” include “instructing positioning of a disc within a sample well.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 hour” includes “1 hour.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure. The phrase “at least one of” is intended to require at least one item from the subsequent listing, not one type of each item from each item in the subsequent listing. For example, “at least one of A, B, and C” can include A, B, C, A and B, A and C, B and C, or A, B, and C.

1. A method for measuring coagulation of a liquid, comprising:
turning on a measuring device,
controlling internal temperature in the device,
inserting a cartridge into the device,
injecting a liquid sample into a well within the cartridge, the well comprising a sidewall and a floor, wherein the well floor is relatively hydrophilic, and the well wall is relatively hydrophobic with respect to the well floor;
providing a contactless magnetic coupling,
reciprocating the well or a movable device within the well with the magnetic coupling,
illuminating the movable device,
observing the movable device with a video camera, and
recording times of changes in movement of the movable device.

2. The method of claim 1, wherein the reciprocating comprises reciprocating the movable device with a contactless magnetic coupling, and the recording comprises recording time differentiation between a start of movement of the movable device and slowing and stopping of movement of the movable device.

3. The method of claim 2, further comprising placing the movable device in the well after the injecting of the liquid sample.

4. The method of claim 1, wherein the turning on comprises connecting a power source to the heat controller and to a motor for the reciprocating, and starting the illuminating and a video camera and a central processor for recording times of changes in movement of the movable device and creating displays according to the changes in movement of the movable device.

5. The method of claim 4, wherein starting the illuminating, the video camera and the central processor comprises turning on a smartphone connected to the measuring device.

6. A method for measuring coagulation of a sample, comprising:
activating a measuring device;
inserting a cartridge into the measuring device;
placing a liquid sample into a well within the cartridge, the well comprising a well wall and a well floor, the well further comprising a disc, the disc comprising a first tracking point comprising a first color, the first tracking point proximate a rotational center of the disc, the disc also comprising a second tracking point comprising a second color spaced apart from the rotational center of the disc, the first color different from the second color, wherein the well floor is relatively hydrophilic, and the well wall is relatively hydrophobic with respect to the well floor.
activating a magnetic field of the measuring device;
rotating the disc in a first direction using the magnetic field;
rotating the disc in a second direction opposite the first direction using the magnetic field;
illuminating the disc;
tracking the first tracking point and the second tracking point of the disc with a video camera; and
calculating changes in movement of the second tracking point with respect to the first tracking point of the disc with a processor to determine coagulation parameters.

7. The method of claim 6, wherein calculating changes in movement of the first tracking point and the second tracking point of the disc occurs in real time.

8. The method of claim 6, wherein the magnetic field comprises a contactless magnetic coupling.

9. The method of claim 6, further comprising displaying the coagulation parameters on a display.

10. The method of claim 9, wherein the display is a smartphone display.

11. The method of claim 6, wherein rotating the disc in a first direction comprises rotating the disc 4° 45′ degrees over 10 seconds.

12. The method of claim 6, further comprising controlling an internal temperature in the measuring device;

13. The method of claim 6, wherein the disc further comprises a spindle, such that the disc is spaced apart from a floor of the well.

14. The method of claim 6, wherein the disc comprises ferromagnetic material to facilitate rotating the disc in the first direction using the magnetic field.

15. The method of claim 6, wherein tracking comprises tracking a reduction in motion of the second tracking point with respect to the first tracking point as the magnetic field becomes no longer strong enough to overcome viscoelasticity of the liquid sample as the liquid sample coagulates.

16. A method for measuring coagulation of a sample, comprising:
activating a measuring device;
inserting a cartridge into the measuring device;
placing a liquid sample into a well within the cartridge, the well comprising a well wall and a well floor, the well further comprising a disc, the disc comprising a first tracking point comprising a first color, the first tracking point proximate a rotational center of the disc, the disc also comprising a second tracking point comprising a second color spaced apart from the rotational center of the disc, the first color different from the second color, the disc spaced apart from a well floor via a spindle operably connected to the disc, wherein the well floor is relatively hydrophilic, and the well wall is relatively hydrophobic with respect to the well floor;
controlling an internal temperature of the measuring device;
activating a magnetic field of the measuring device;
rotating the disc in a first direction using the magnetic field;
rotating the disc in a second direction opposite the first direction using the magnetic field;
illuminating the disc;
tracking the first tracking point and the second tracking point of the disc with a camera; and
calculating changes in movement of the second tracking point with respect to the first tracking point of the disc with a processor to determine coagulation parameters,
wherein tracking comprises tracking a reduction in motion of the second tracking point with respect to the first tracking point over time as the magnetic field becomes no longer strong enough to overcome viscoelasticity of the liquid sample as the liquid sample coagulates.

17. The method of claim 16, wherein the well floor comprises a hydrophilic coating.

18. The method of claim 16, wherein the well wall comprises a hydrophobic coating.

19. The method of claim 16, wherein the disc is positioned substantially parallel to the well floor.

20. The method of claim 16, wherein a disc diameter to well diameter can be between about 0.5 and about 1.0.

21-35. (canceled)
